(19) United States
(12) Patent Application Publication
(10) Pub. No.: US 2026/0250310 A1
(43) Pub. Date: Aug. 27, 2026

(54) Abiotic Catalytic Conversion of Carbon Dioxide to Sugars

(71) Applicant: The Regents of the University of California, Oakland, CA (US)

(72) Inventors: Peidong Yang, Berkeley, CA (US); Jie Luo, Berkeley, CA (US); Nathan Edward Soland, Berkeley, CA (US)

(73) Assignee: The Regents of the University of California, Oakland, CA (US)

(21) Appl. No.: 19/651,570

(22) Filed: Apr. 17, 2026

Related U.S. Application Data
(63) Continuation of application No. PCT/US24/51870, filed on Oct. 18, 2024.
(60) Provisional application No. 63/592,135, filed on Oct. 20, 2023.
(60) Provisional application No. 63/677,407, filed on Jul. 31, 2024.

(51) Int. Cl. C07H 3/02; B01J 27/04; B01J 31/02; B01J 35/39; C25B 3/07; C25B 3/26; C25B 9/19; C25B 11/032; C25B 11/043

(52) U.S. Cl. CPC C07H 3/02; B01J 27/04; B01J 31/0235; B01J 35/39; C25B 3/07; C25B 3/26; C25B 9/19; C25B 11/032; C25B 11/043

(57) ABSTRACT

Methods, devices and systems are used for abiotic catalytic conversion of carbon dioxide to a sugar, formaldehyde-to-sugar, formaldehyde-to-fuel or methanol-to-sugar.

### CROSS-REFERENCES TO RELATED APPLICATIONS

This application claims priority to U.S. Ser. 63/677,407, filed: Jul. 31, 2024 and US Ser. 63/592,135; filed: Oct. 20, 2023, the disclosures of which are hereby incorporated by reference in its entirety for all purposes.

### INTRODUCTION

There is a surplus of carbon dioxide (CO₂) emissions and a looming deficit of petroleum-derived fuels. Catalytic conversion of CO₂ to valuable chemicals (such as carbon monoxide, methanol, formic acid, acetate, ethanol) with selectivity and high energy efficiency represents a promising approach to address both problems simultaneously (1). Carbohydrates are an immensely valuable target product. Approximately half of the world's caloric needs can be satisfied by carbohydrates (2, 3). Producing artificial sugars directly without extraneous crop components (e.g. structural carbohydrates) and associated externalities has been explored as an approach to address the world's food requirements (2, 4). If deemed unsuitable for human consumption, these artificial sugars may be converted to other valuable molecular targets such as fuels via fermentation or pharmaceuticals and plastics by chemical upgrading or microbial manufacturing (1, 5).

Artificial CO₂-to-sugar strategies explored may be classified as biotic and abiotic (6, 7). Metabolic engineering and chemoenzymatic pathways have been broadly explored for improving CO₂ conversion to carbohydrates, taking advantage of simple two electron reduction pathways for CO₂ fixation or existing thermocatalytic technologies combined with the powerful stereoselectivity of enzymatic reactions to produce carbohydrates (6, 8-11). However, these chemoenzymatic concepts suffer from many of the same challenges of biology, including low titer of product, slow turnover, intolerance to formaldehyde as an intermediate in C₁ conversion, and expensive enzymatic engineering and isolation. Furthermore, although biology is highly efficient as individual steps, in total the photosynthesis of biomass falls short of ideal solar-to-chemical conversion efficiency, generally only approaching 1% (12, 13).

High carbon conversion rate in a minimum number of abiotic steps, even at lower stepwise efficiency and selectivity, may be comparable to the solar-to-chemical conversion potential of conventional crops and have scalability potential (2, 14). For land allocation in a biofuel-centric economy, these metrics are crucial. However, significant research advancements are required for each necessary catalytic step in CO₂ upgrading to carbohydrates. Current abiotic approaches focus on using formaldehyde to generate various carbohydrates and side products via the formose reaction (7, 15). Unfortunately, selective production of formaldehyde from CO₂ lags behind that of simpler products such as carbon monoxide. Additionally, control of C₁ condensation chemistry has limited progress. We therefore aimed to address each of these tasks and pursued the development of a constrained strategy to convert CO₂ to CH₂O using only sustainable electro- or photo-catalysis followed by low-temperature organocatalytic conversion to Cₙ monosaccharides (C₃₋₆).

### SUMMARY OF THE INVENTION

In aspects the invention provides a modular approach for carbon dioxide (CO₂) to multicarbon (Cₙ) upgrading for commodity chemicals, fuel production or artificial food synthesis, utilizing sequential electro-, photo- and organocatalysis. In embodiments, we employ the electrochemical carbon dioxide reduction reaction (CO₂RR) to methanol in a flow cell and its selective photooxidation to formaldehyde (PMOR), which is the C₁ precursor of interest for sugar generation. Utilizing an active N-heterocyclic carbene (NHC) catalyst enables tunable generation of C₃-C₆ aldoses without undesirable byproducts, with carbon conversion yield reaching 60-80% for desired pentose, tetrose and triose product mixtures and over 20% for hexose. The demonstrated catalytic strategies offer a useful direction for CO₂ upgrading to valuable chemicals.

In aspects the invention provides a two-fold strategy for selective generation of sugar from CO₂: first, a (photo) electrochemical reduction strategy in which formaldehyde is first generated from CO₂, or CO₂-derived methanol with partial oxidation faradaic efficiency typically exceeding 50%; and second, selective sugar generation from formaldehyde using N-heterocyclic carbene chemistry, capable of selective condensation reactions. Selectivity for the formaldehyde-to-sugar transformation typically exceeds 80% toward desired pentose, tetrose, triose, and hexose products, and the carbon utilization is practically quantitative, representing an efficient and effective strategy for carbon upcycling.

In aspects the invention provides:
A method comprising abiotic catalytic conversion of carbon dioxide to a sugar, formaldehyde-to-sugar, formaldehyde-to-fuel or methanol-to-sugar.
A device or system configured for abiotic catalytic conversion of carbon dioxide to a sugar, formaldehyde-to-sugar, formaldehyde-to-fuel or methanol-to-sugar.
A method, device or system herein for production of xylose, glucose, dihydroxyacetone, erythrose, furfural derivatives, etc., particularly as synthesized from formaldehyde using N-heterocycle carbenes (NHCs) methods.
A method, device or system herein, substantially as depicted in the figures or schemes.

In aspects and embodiments the invention provides:
1. A method for selective generation of a sugar from carbon dioxide, comprising: (a) catalytically converting carbon dioxide (CO₂) to formaldehyde (CH₂O) via (i) reduction of CO₂ to methanol (CH₃OH) followed by oxidation to CH₂O, or (ii) direct reduction of CO₂ to CH₂O; and (b) using an N-heterocyclic carbene catalyst to selectively condense the formaldehyde to generate the sugar.
2. The method of claim 1 wherein step (a) comprises electrochemical reduction of the CO₂ to methanol in an electrochemical carbon dioxide reduction reaction (CO₂RR), and photochemical oxidation of the methanol to formaldehyde in a photooxidation of methanol to formaldehyde oxidation reaction (PMOR).
3. The method of claim 1 wherein step (a) comprises electrochemical reduction of the CO₂ to methanol on a catalyst comprising carbon nanotube (CNT)-supported cobalt (II) phthalocyanine (CoPc).
4. The method of claim 1 wherein step (a) comprises electrochemical reduction of the CO₂ to methanol on a catalyst comprising carbon nanotube (CNT)-supported cobalt (II) phthalocyanine (CoPc) deposited on a support, such as carbon paper, in aqueous or nonaqueous (e.g. acetonitrile) solvent with supporting electrolyte.
5. The method of claim 1 wherein step (a) comprises electrochemical reduction of the CO₂ to methanol using a membrane electrode assembly (MEA) flow cell architecture, such as a gas diffusion electrode to enhance the transport of CO₂ to the catalyst for increased product yield.
6. The method of claim 1 wherein step (a) comprises electrochemical reduction of the CO₂ to methanol using an H-cell architecture to increase a faradaic efficiency.
7. The method of claim 1 wherein step (a) comprises partial oxidation of CH₃OH to CH₂O using a noble metal catalysts, such as platinum and palladium, capable of electrooxidation of CH₃OH.
8. The method of claim 1 wherein step (a) comprises partial oxidation of CH₃OH to CH₂O using a noble metal catalysts, such as platinum and palladium, capable of electrooxidation of CH₃OH, and tuning reaction parameters, including methanol content, to improve selectivity to CH₂O over products such as CO and CO₂, improving space-time yield by utilizing a flow cell architecture which enhances the convective removal of formaldehyde product from the electrode surface.
9. The method of claim 1 wherein step (a) comprises electrochemical reduction of the CO₂ to methanol, wherein faradaic efficiency, a metric describing the fraction of electric current utilized for generation of the desired product, exceeds 50% in an H-cell configuration on palladium foil.
10. The method of claim 1 wherein step (a) comprises electrochemical reduction of the CO₂ to methanol, wherein partial current densities to formaldehyde approach the milliamp per square centimeter range depending on overpotential applied, methanol concentration and solvent composition, solution pH, convective conditions in the cell, and electrode pretreatment.
11. The method of claim 1 wherein step (a) comprises electrochemical reduction of the CO₂ to methanol (CH₃OH) followed by oxidation to CH₂O, wherein photocatalytic oxidation of CH₃OH to CH₂O comprises selecting photocatalysts with suitable band edge positions and stability for extended operation.
12. The method of claim 1 wherein step (a) comprises electrochemical reduction of the CO₂ to methanol (CH₃OH) followed by oxidation to CH₂O, wherein photocatalytic oxidation of CH₃OH to CH₂O comprises selecting photocatalysts with suitable band edge positions and stability for extended operation, in an implementation utilizing zinc-indium-sulfide (ZIS) nanostructures under UV or broad-spectrum irradiance.
13. The method of claim 1 wherein step (a) comprises electrochemical reduction of the CO₂ to methanol to yield reaction products including hydrogen (H₂), carbon monoxide (CO), CH₂O, and methanol (CH₃OH), the method further comprising adjusting relative selectivity and yield of these products by tuning catalyst loading, solution pH, supporting electrolyte, gas feed rate, applied potential, temperature, and electrolyzer design.
14. The method of claim 1 wherein step (b) comprises preactivated NHC in an aprotic, nonpolar solvent such as dioxane, wherein there is little driving force for ketose formation via isomerization, and the condensation reaction can proceed until ring formation terminates the reaction differentially based on reaction temperature and composition, wherein higher order carbohydrates are afforded without byproducts from the classical formose reaction.
15. The method of claim 1 wherein in step (b) NHC formose catalysis is tuned further by employing a diverse range of NHCs, including enantiomerically selective catalysis more stability afforded by modifying the steric hindrance at the carbene center, allowing for implementation in aqueous solvent; or more reactivity is afforded by replacing the substituents; or enhanced product separation is achieved by immobilization strategies of the catalysts onto polymers or silica gel.
16. The method of claim 1 wherein step (a) comprises direct reduction of CO₂ to CH₂O using a boron doped diamond catalyst.
17. The method of claim 1 wherein step (a) comprises direct reduction of CO₂ to CH₂O using a boron doped diamond catalyst, utilizing an aqueous or mixed nonaqueous (e.g. acetonitrile/water) electrolyte in an H-cell or membrane electrode assembly (MEA) flow cell architecture, such as a gas diffusion electrode, for direct electrosynthesis of CH₂O from CO₂.
18. The method of claim 1 wherein step (a) provides a partial oxidation faradaic efficiency exceeding 50%;
19. The method of claim 1 wherein selectivity for the formaldehyde-to-sugar transformation exceeds 80% toward desired pentose, tetrose, triose, and hexose products,
20. The method of claim 1 wherein carbon utilization is practically quantitative, representing an efficient and effective strategy for carbon upcycling.
21. The method of claim 1 wherein the sugar is a C₃-C₆ aldose.
22. The method of claim 1 wherein step (b) generates the sugar without oxidized byproducts from a classical formose reaction
23. The method of claim 1 wherein step (b) comprises organocatalytic benzoin-like condensation of formaldehyde catalyzed by N-heterocyclic carbene (NHC) to preferentially form aldose and unbranched monosaccharides, distinguishing this approach from uncontrolled alkaline formose.
24. The method comprising in situ generation and utilization of the formaldehyde to minimize processing steps.
25. The method of claim 1 further comprising isolating the sugar.

The invention encompasses all combinations of the particular embodiments recited herein, as if each combination had been laboriously recited.

### BRIEF DESCRIPTION OF THE DRAWINGS

FIG. 1. Process overview for carbon dioxide conversion to carbohydrates (sugars) using electrocatalytic, photocatalytic, and organocatalytic strategies.

FIG. 2. Process overview with preferred strategies and benefits highlighted.

FIG. 3. Electrocatalytic oxidation of generated methanol over noble metal catalyst (foil or nanoparticles).

FIG. 4. Scheme of photocatalytic oxidation of formaldehyde using Zinc Indium Sulfide (ZIS) semiconductor photcatalysis with cogeneration of hydrogen.

FIGS. 5A-B. N-Heterocyclic carbene catalyzes aldose generation from formaldehyde. (A) Mechanistic scheme of NHC-catalyzed formoin condensation. (B) Simplified scheme showing formoin reactions leading to generation of stable tetrose, pentose, and hexose products.

FIG. 6. Characterization and electrocatalytic CO₂RR performance of immobilized cobalt phthalocyanine (CoPc) on carbon nanotubes (CNTs). Schematic of electrochemical flow cell and associated reactions.

FIG. 7. Characterization and photocatalytic MOR performance of zinc indium sulfide (ZIS) nanocrystals. Schematic showing the carrier diffusion, charge separation and Ni-dependent dehydrogenation of methanol to formaldehyde.

FIG. 8. Formose reaction mechanism for comparison to formoin. The alkaline environment promotes undesirable side reactions such as dehydration, Cannizzaro disproportionation, and coupling to produce branched sugars.

FIG. 9. Flowchart indicating accessible aldoses from formaldehyde using the formoin reaction, including some indication of market value and theoretical yield assuming C₁ addition to the precursor directly above, some applications. Successive C₁ additions generate, in essence, two possible diastereomers. For each class of monosaccharide, the total selectivities from NHC-1 are indicated, along with the individual stereoisomer yield from formaldehyde on the mathematical basis coupled with experimental result, and from CO₂ by factoring in catalytic selectivities. Note: only D-configurations are depicted, though the NHC will generate racemized product.

FIG. 10. Colloidal synthesis scheme for ZIS.

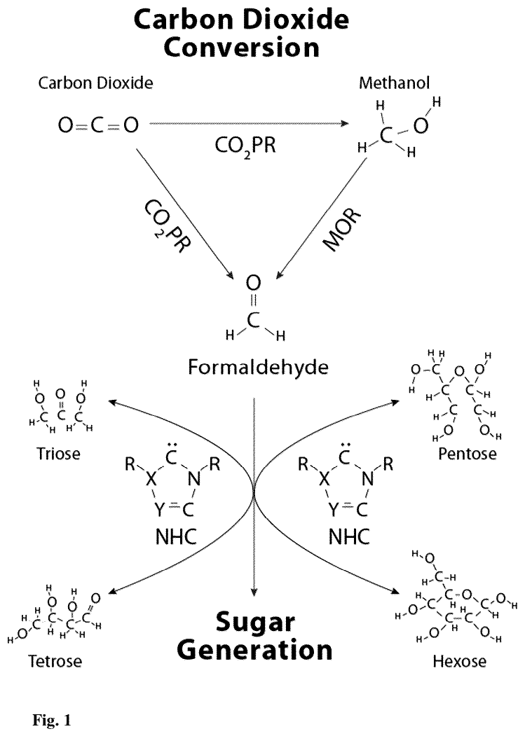

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The invention provides a process comprising two aspects: the generation of formaldehyde (“Part 1,” or Carbon Dioxide Conversion) and the utilization of formaldehyde to produce multicarbon species (“Part 2,” Sugar Generation).

### Part 1 Background

The generation of formaldehyde from carbon dioxide is technologically feasible with existing thermal catalytic technologies. CO₂ can be directly reduced to methanol on the pilot plant scale, and methanol thermal oxidation (as in the BASF oxidation process) is well-established for producing aqueous solutions of formaldehyde. However, thermal catalytic approaches require high temperature and pressure to operate, and thus are currently impractical for electrification.

A strategy employing electrocatalysis or photocatalysis would be ideal for greener process chemistry due to the suitability for electrification. The invention provides a process in which direct electro- or photoreduction of carbon dioxide to formaldehyde, or electro- or photoreduction to methanol followed by partial oxidation to formaldehyde, is employed for generation of the formose precursor.

### Part 1 Methods

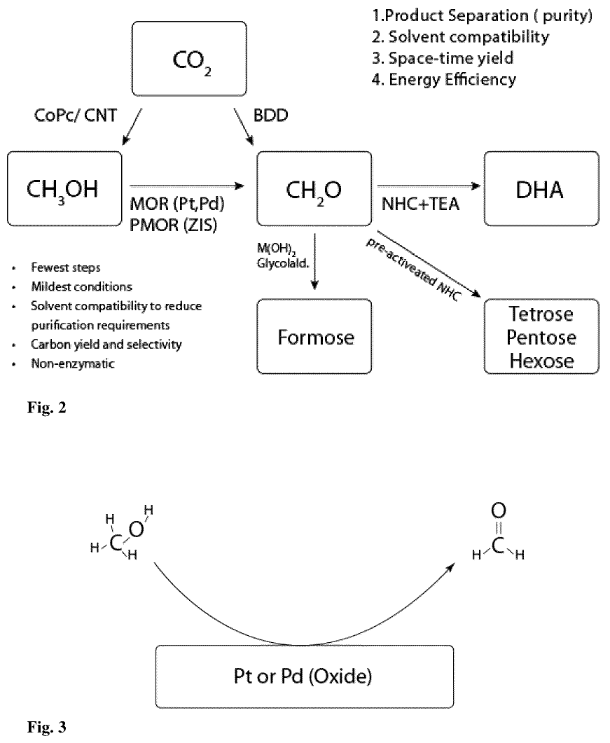

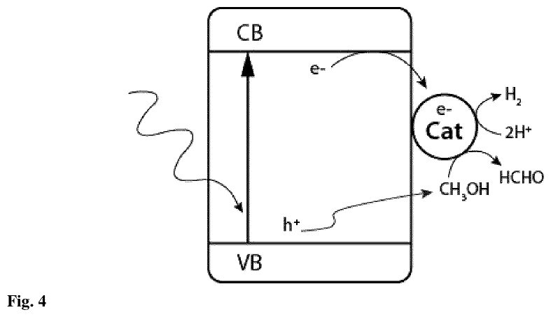

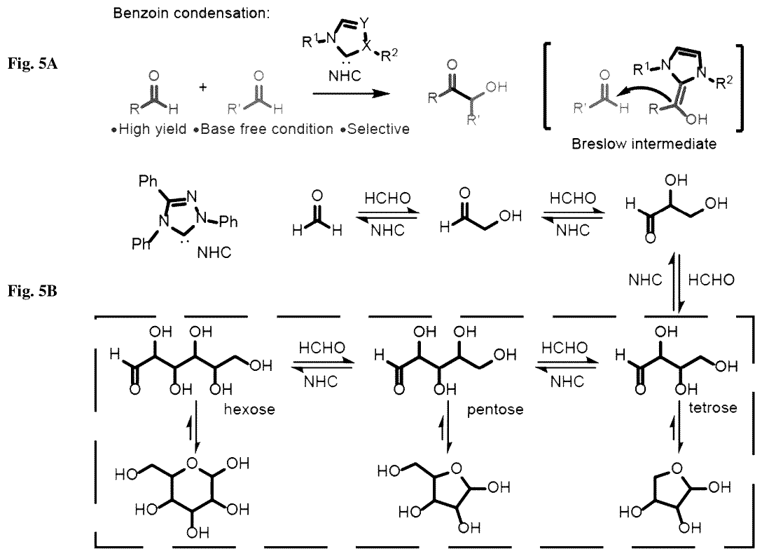

The choice of catalyst identity and operating conditions have a significant impact on yield and efficiency. A process of the invention employs at least one of several methods, depicted in FIG. 2. In summary, carbon dioxide (CO₂) is catalytically converted to formaldehyde (CH₂O) via one of several methods. The approach of Part 1 is divided into two paths: (A) reduction of CO₂ to CH₃OH followed by oxidation to CH₂O, and (B) direct reduction of CO₂ to CH₂O.

(A): CO₂ can be electroreduced on catalysts including carbon nanotube (CNT)-supported cobalt (II) phthalocyanine (CoPc). In aqueous electrolyte, the dominant products include hydrogen (H₂), carbon monoxide (CO), CH₂O, and methanol (CH₃OH). The relative selectivity and yield of these products can be influenced by tuning catalyst loading, solution pH, supporting electrolyte, gas feed rate, applied potential, temperature, and electrolyzer design. Electrolyte may be aqueous, consisting solely of water and supporting electrolyte, or nonaqueous, consisting of an organic solvent and supporting electrolyte, or mixed aqueous, a ternary system comprising water, organic solvent, and supporting electrolyte. A preferred implementation comprises CoPc/CNT deposited onto a support, such as carbon paper, in aqueous or nonaqueous (acetonitrile) solvent with supporting electrolyte. A membrane electrode assembly (MEA) flow cell architecture, such as a gas diffusion electrode, is preferred for enhancing the transport of CO₂ to the catalyst for increased product yield. An alternative implementation utilizes an H-cell architecture. With aqueous H-cell tests, CO₂ reduction to methanol reaches a faradaic efficiency of 4-5% without optimization, greater than 10, 20 or 30% with optimization.

The partial oxidation of CH₃OH to CH₂O can be accomplished with several strategies. Noble metal catalysts, such as platinum and palladium, are capable of electrooxidation of CH₃OH. By tuning methanol content in addition to the parameters listed above, the selectivity to CH₂O over products such as CO and CO₂ can be achieved. Improved space-time yield may be accomplished by utilizing flow cell architectures which enhance the convective removal of formaldehyde product from the electrode surface. Typical faradaic efficiency, a metric describing the fraction of electric current utilized for generation of the desired product, exceeds 50% in an H-cell configuration on palladium foil. Partial current densities to formaldehyde may readily approach the milliamp per square centimeter range depending on overpotential applied, methanol concentration and solvent composition, solution pH, convective conditions in the cell, and electrode pretreatment.

The photocatalytic oxidation of CH₃OH to CH₂O can be accomplished by selecting photocatalysts with suitable band edge positions and stability for extended operation. One implementation is achieved with zinc-indium-sulfide (ZIS) nanostructures under UV or broad-spectrum irradiance, with production rate reaching around 5000 μmol g⁻¹ h⁻¹. A benefit of the photocatalytic system implementation is the catalyst's solvent agnosticism, enhancing total system integration, as well as dispersion as a suspension, for improved CH₃OH utilization at low concentrations.

(B): Another catalyst for direct CO₂ reduction to CH₂O is highly boron-doped diamond. This catalyst may be generated via chemical vapor deposition methods and similar electrocatalytic optimization strategies apply, with additional tuning parameters including boron dopant concentration, diamond grain size and graphitic content. A preferred implementation of this approach utilizes aqueous or mixed nonaqueous (acetonitrile/water) electrolyte in an H-cell or MEA configuration for direct electrosynthesis of CH₂O from CO₂. Benefits of this approach are the stability of diamond-derived materials for extended catalytic durations, nontoxic catalyst components, suitability for multiple electrolyte compositions and electrolyzer designs, and reduced catalytic steps required for completion of Part 1, which enhances energy efficiency of the CO₂-to-sugars process.

### Part 2 Background

The alkaline formose reaction is generally nonselective, yielding a range of C₂-C₈ and higher sugars, acids, and alcohols. Preferred process design requires carbon utilization toward a desired product. An approach that is capable of producing Cₙ carbohydrates (n=3 triose, n=4 tetrose, n=5 pentose, n=6 hexose) on demand would take a large step toward technoeconomic utility in CO₂ valorization, for instance.

N-heterocycle carbenes (NHCs) are a useful class of molecules which contain nitrogen atoms within a ring structure and a stable carbene carbon. A large number of NHCs have been synthesized due to the diverse structural forms that are capable of stabilizing the carbene atom. Due to the strong nucleophilicity of these carbenes, NHCs are capable of acting as highly Lewis basic organocatalysts for a number of useful organic reactions, ranging from transesterification to polymerization reactions.

We employ a novel pre-activation strategy in which the NHC carbene is not generated in situ but prior to reaction with t-BuOK. This modification, along with solvent, temperature, catalyst loading, and reaction time optimization, enables selective generation of groups of multicarbon products, including triose, tetrose, pentose, and hexose with avoidance of side products.

### Part 2 Methods

Reactions were performed in a number of aqueous and nonaqueous organic solvents, including DMF, MeCN, diglyme, THF, dioxane, 2-methoxyethanol, dichloroethane, dibutyl ether, methanol, and water. Briefly, all reactions are performed in air-free conditions. The NHC is added to the reaction flask. The requisite amount of formaldehyde is added to the evacuated and argon-flushed flask, followed by solvent. The flask is sealed and heated at the desired temperature and reaction time for sugar production.

For generation of tetrose, lower temperature, lower NHC loading, and shorter reaction times are preferred. In one implementation, 3 mg of NHC 1 was added to an argon-purged reaction flask. Paraformaldehyde was suspended in p-dioxane. The sealed flask was heated to 80° C. for several hours. The product was cooled, dried on a rotary evaporator, and collected for gas chromatographic analysis.

For generation of pentose, higher temperature (90-110° C.), lower NHC loading, and shorter reaction times are preferred. In one implementation, 3 mg of NHC 1 was added to an argon-purged reaction flask. Paraformaldehyde was suspended in p-dioxane. The sealed flask was heated to 90-100° C. for several hours. The product was cooled, dried on a rotary evaporator, and collected for gas chromatographic analysis.

For generation of hexose, higher temperature, higher formaldehyde concentration and higher NHC loading is preferred. Reaction temperature ranging 100-110° C. with three times higher NHC loading as in the pentose case is suitable for a marked difference in hexose yield.

For generation of triose (especially 1,3-dihydroxyacetone, DHA), preferred conditions are similar to those used in previous studies. Uniquely, in situ activation of the thiazole based NHC catalyst leads to the termination of reaction at DHA.

Products were detected and quantified by acetylation followed by gas chromatography. Sorbitol (0.2 mmol) was employed as a convenient internal standard for the quantification. The sugar product was dried and dissolved in pyridine with vigorous stirring, followed by dropwise addition of acetic anhydride yielding (after several hours) volatile-OAc protected carbohydrate species for gas chromatographic separation. Product traces were compared with commercial carbohydrate standards. Secondary confirmation was obtained using high performance liquid chromatography.

### Part 2 Results in Brief

The pre-activated NHC organocatalysis was optimized based on reaction temperature, reaction time, choice of solvent, catalyst loading, formaldehyde concentration, and NHC identity. In our general results tuning to Cₙ (n=3-6) was demonstrated.

Notably, the generation of sugar acids and alcohols, omnipresent in the base-catalyzed formose and organic condensation reactions, is largely and effectively suppressed with this method, affording improved carbon yield toward desired sugar products, as shown in distinct grouping of products by mass in the chromatogram traces. The distinct benzoin-like condensation also preferentially forms aldose and unbranched monosaccharides, again distinguishing this approach from the uncontrolled alkaline formose.

Catalyst loading was shown to have a marked effect on the sugar selectivity. Higher relative loading of NHC 1 caused significant increase in hexose yield, to a limit of around 30%.

To isolate the carbohydrate product we first dried the product in a rotary evaporator. The NHC and sugar product was obtained as a light yellow product. The NHC 1 catalyst was removed by sonication of the product in diethyl ether, leaving a white residue consisting primarily of the sugar compositions. The resulting white product was purified by antisolvent recrystallization. The sugars were dissolved into a minimum volume of warm ethanol before adding cold diethyl ether, which immediately precipitated the majority of sugars. After storing at −10° C., yielding a white suspension, which upon drying is between a viscous, sticky syrup and a hard solid, depending on relative ratios of carbohydrates. Dry mass yield of carbohydrate product was approximately 70-80% in optimized conditions based on initial formaldehyde content.

### Integrating Part 1 and 2

The spatiotemporal separation of the two aspects of this process enables significant flexibility, depending on desired considerations for the full system, including:
1) Minimal processing steps: the technoeconomic utility of the process is more favorable with decreased requirements for separate purification steps, fewer reactions, higher atom economy.
2) Energy efficiency: the utility of the process is more favorable with an overall reduction in energy usage per step, and fewer steps overall.

To minimize processing steps, the in situ generation and utilization of formaldehyde is preferred. Solvent compatibility for solution-based chemical reaction steps is preferred. Full system consideration of the choice of electrolyte and choice of NHC formose solvent indicates the following integration methods: (A) Use of aqueous electrolyte for CO₂ reduction implies the use of NHC catalyst that is stable in aqueous environment. (B) The use of NHC that is stable in aprotic, nonaqueous solvent implies the use of a compatible nonaqueous electrolyte for CO₂ reduction. (C) The use of an aqueous electrolyte for CO₂ reduction, in the absence of a suitably stable NHC, implies the consideration of:
i. Separation of CH₂O from the resulting solution post-catalysis in Part 1. This may be achieved by distillation techniques or freezing/filtration.
ii. The classical aqueous formose reaction. This is a simpler way to generate multicarbons, but lacks the specificity afforded by the NHC formose reaction.

To improve the energy efficiency of the reaction and carbon atom economy, it is preferable to minimize catalytic steps, lower the energy requirements of each step, and improve the carbon utilization for each step. These considerations indicate the following implementations: (A) The use of NHC catalysts to improve carbon utilization for desired carbohydrate products; (B) The use of direct CO₂ reduction to CH₂O; (C) The use of CO₂ reduction to CH₃OH followed by efficient oxidation to CH₂O.
i. For low concentrations of generated methanol, photocatalytic oxidation allows for dispersion and high selectivity toward formaldehyde.
ii. For high concentrations of generated methanol, or in nonaqueous electrolyte with low concentrations of methanol, electrocatalytic oxidation allows for higher reaction rates by tuning of catalyst potential without the tendency toward highly oxidized byproducts.

The multipronged approach (electrocatalysis and photocatalysis, aqueous and nonaqueous) allows for flexibility regarding the aforementioned parameters. Additional embodiments may include implementation of the aforementioned aspects and optimization of each component to produce a process workflow that is energy efficient and produces the desired product with selectivity and ease of isolation.

### Innovations

Electro/photogeneration of formaldehyde from CO₂ represents a marked step toward improving the environmental impact of formaldehyde production. The soluble formaldehyde product is readily integrated into the sugar generation step in Part 2.

By using a preactivated NHC in an aprotic, nonpolar solvent such as dioxane, there is little driving force for ketose formation via isomerization, and the condensation reaction can proceed until ring formation terminates the reaction differentially based on reaction temperature and composition. Thus, higher order carbohydrates are afforded without byproducts from the classical formose. The NHC formose reaction affords greater control over product selectivity than the classical formose and is a practical component a total system generating carbohydrates on demand from carbon dioxide.

Previous ideations of the CO₂-to-sugars pathway have employed only the classical formose reaction, or devised multistep, chemoenzymatic strategies for targeting hexose sugars. This electro/photocatalytic strategy coupled with NHC-driven organocatalysis is a distinct manifestation of the concept with preferable simplicity, selectivity, mild reaction conditions, and good yield.

### ADDITIONAL EMBODIMENTS

NHC formose catalysis may be tuned further by employing a diverse range of NHCs, including enantiomerically selective catalysis more stability afforded by modifying the steric hindrance at the carbene center, potentially allowing for implementation in aqueous solvent; more reactivity afforded by replacing the substituents; enhanced product separation by immobilization strategies of the catalysts onto polymers or silica gel.

### Examples: Multi-Step Catalytic Abiotic CO2 to Cₙ Sugars Through C1 Intermediates

In these examples, we demonstrate lab-scale conversions of carbon through the C₁ to Cₙ modules to explore feasibility of complex CO₂ conversion in individually optimized steps. For electrocatalytic CO₂ reduction we achieve 35 mA cm⁻² (217 μmol hr⁻¹ cm⁻²) production of methanol. Photocatalytic conversion to formaldehyde (PMOR) reaches a stable methanol conversion rate of 20 mmol per gram of photocatalyst per hour (g cat⁻¹ hr⁻¹). The transformation of C₁ to Cₙ carbohydrates approaches high batch conversion rates at the gram scale. The biological compatibility of NHC-derived sugars is demonstrated using E. Coli, which had a specific growth rate of 0.138 hr⁻¹ in M9 minimal media with NHC sugars as the sole carbon source. We aim to highlight advantages and current limitations of the modular strategy, describe target efficiencies, and enable broad-domain collaboration and integration for sustainable catalysis.

Organocatalysis for Selective Cₙ Generation from Formaldehyde

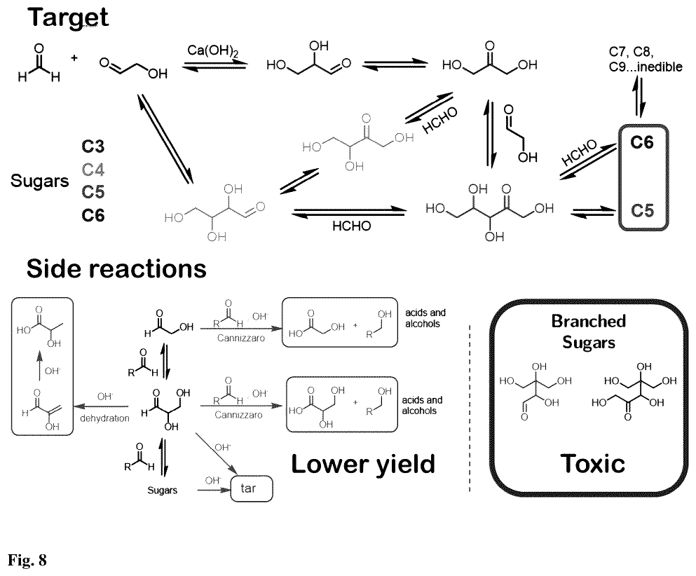

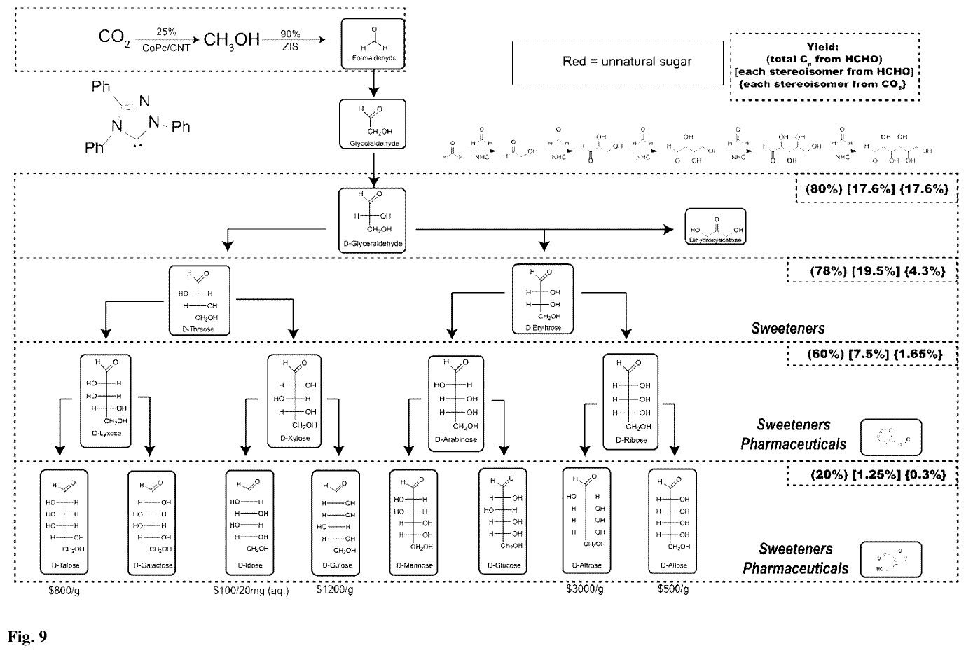

The formose reaction has long been pursued to uniquely produce complex carbohydrates from formaldehyde in an alkaline environment. However, kinetic instabilities associated with its autocatalytic mechanism preclude precise control of the product distribution and inevitably reduce carbon yield of carbohydrates (FIG. 8). This creates an intractable separation task and consequent technoeconomic prohibition, despite decades of research efforts (16). Significant product simplification is necessary for a practical C₁ to Cₙ process. To this end, organocatalytic benzoin-like condensation of formaldehyde catalyzed by N-heterocyclic carbenes (NHCs) may be considered as a promising alternative approach, which ideally can assemble multiple formaldehyde molecules into higher sugars without generating copious waste products (FIG. 5A-B) (17-19). The abiotic CO₂-to-sugars vision may incorporate this strategy due to the advent of greater tunability than the uncontrolled formose reaction.

The use of formaldehyde (CH₂O) as a substrate for NHC-catalyzed condensation reactions has been demonstrated, but generally leads to C₂-C₄ short-chain carbohydrate products (17, 18, 20). Generation of pentose and hexose has been observed for sufficiently high concentration of base, high temperatures and a thiazolium catalyst (20). However, tuning of conditions to controllably generate different classes of aldose product with low catalyst loading and high carbon conversion efficiency has not been attained. Consequently, we sought to extend its utility for producing versatile saccharide products. Ender's carbene NHC-1 (1,3,4-triphenyl-4,5-dihydro-1,2,4-triazol-5-ylide, FIG. 5B) was used as the catalyst for our attempts due to its excellent stability under heating, which is essential for the generation of higher sugars. In addition, instead of using the conventional cationic carbene precursor which needs to be activated by the inclusion of base, we utilized an alcohol-trapped carbene precursor (1,3,4-triphenyl-4,5-dihydro-5-methoxy-1,2,4-triazole) (18), which generates the active carbene species in situ by heating during catalysis without need for an activating base (21). The exclusion of additional base is crucial for selective and facile generation of aldoses beyond C₃ and rationally accounts for the selectivities obtained.

We systematically optimized the temperature, reaction time, and solvent to control the degree of CH₂O condensation, aiming to generate different speciation of sugars. General trends observed include the ability to selectively generate tetrose, pentose, and some hexose sugars, which can form thermodynamically favorable furanose or pyranose structures (FIG. 5B). These structures exhibit thermodynamic differences among the various products, providing opportunities to halt the reaction at the generation of each product through precise control of the reaction conditions (Table 1). Gas chromatography (GC) of acetylated products in conjunction with HPLC, 1H-NMR, 13C-NMR and IR confirm the presence of desirable high sugars including lyxose, xylose, arabinose, ribose, glucose and galactose. In the optimized reactions, carbon yield of tetrose, pentose and hexose were 78, 62 and 23% respectively, as calculated by GC analysis of the acetylated product by calibration with a sorbitol internal standard. In general, fast reactivity accomplished by higher catalyst loading and higher temperature favors higher-carbon products.

To explore the unique tunability of the formoin reaction between C₄-C₆ and its integration with CO₂ catalysis processes, we tested how various solvent conditions influence selectivity. The use of an aprotic solvent such as p-dioxane was beneficial for reactivity, likely due to its good solubility for the generated polyol intermediates and its ability to preserve the reactive Breslow intermediate species (FIG. 5A). Use of a polar protic solvent such as methanol or water inhibits the reaction due to protonation of the carbene site. Productivity was diminished in polar aprotic solvents such as DMF and MeCN (Table 1). This presents a challenge to direct one-pot integration with electrocatalytically and photocatalytically generated formaldehyde, which typically requires aqueous and polar solvents, but is readily overcome by isolation of the formaldehyde product or its surrogate.

Selective generation of primarily aldose sugars merits some additional explanation. Once formaldehyde is converted to glycolaldehyde (C₂), there are by definition C_{>1} reactants available for the NHC to utilize, which may be expected to complicate the product mixture. For example, ketose generation is also possible through C₂-C₂₊ coupling and is indicated by the presence of additional peaks of intermediate retention time in the GC chromatogram. Indeed, NHC-1 can also utilize glycolaldehyde (C₂H₄O₂) as the initial substrate and displays these intermediate peaks. Yet in the C₁ formoin reaction, we do not generally observe these species. The generation of C₅ species from glycolaldehyde indicates the presence of retro-benzoin reactions to form HCHO, which then undergoes the typical formoin reaction, and eventually reaches stabilized furanose and pyranose products (C₄-C₆ sugars). This indeed increases the product complexity, but we posit that in the presence of formaldehyde, the direct coordination of NHC-1 to the higher aldehydes is sterically disfavored, suppressing the generation of the byproducts observed in the glycolaldehyde reaction and improving aldose selectivity.

Full accounting for the unique selectivity of the formoin reaction must still acknowledge the possibility of ketose generation and their reactions in conjunction with the C₁ addition mechanism. We found that stabilized ketoses are generally terminal species for the reaction by showing that dihydroxyacetone (DHA, C₃ ketose) and erythrulose (C₄ ketose) are not suitable substrates for the NHC. Using them as the substrates in the presence of NHC-1 yields no further unique products. Therefore, not only is the generation of ketose sugar less favorable, but they are present primarily as spectators, and thus a two-pronged mechanism for suppression of non-aldose products is achieved.

Dual feeding of DHA and formaldehyde with NHC-1 in the absence of TEA produces a product distribution similar to that for formaldehyde alone, with the excess DHA remaining post-reaction as a spectator. In total, this confirms that NHC-1 without additional base is necessary for selective beyond-C₃ product generation and that DHA is unsuitable as a substrate even with formaldehyde coupling. This agrees with previous reports of selective DHA formation using various NHCs and emphasizes the unique strategy of our base-free formoin. Notably, the non-reactivity with DHA suggests that branched sugars, considered toxic byproducts of uncontrolled formose chemistry, cannot be generated from this system. This further highlights the superiority of this method (FIG. 8, FIG. 9).

To explore the applications of the NHC formoin reaction, we scaled up the reaction to gram scale and isolated the carbohydrate product mixture for further purification and feeding to E. coli, a model heterotrophic organism for metabolic engineering and biomanufacturing. After formoin product purification, 83% mass yield was attained as a yellow syrup. The formoin sugars were fed to E. coli as the sole carbon source (1% wt/wt) in M9 minimal media. Cell growth was validated by sigmoidal increase of optical density to a stationary phase of ~ 0.6 with a specific growth rate of 0.138 hr⁻¹, indicating biocompatibility of the formoin product. For comparison, glucose-only substrate was expectedly higher-performing, with a specific growth rate of 0.347 hr⁻¹, indicating lower metabolic availability of C₄ and C₅ sugars.

Electrocatalytic Methanol Generation from CO₂

The potential for using formaldehyde (CH₂O) for controlled multicarbon generation invites the challenge of producing the requisite C₁ from CO₂ in an electrified catalytic system. Despite major progress in heterogeneous electrocatalysis in the last decade, directing multiple sequential proton-coupled electron transfers is kinetically challenging and thus a major research challenge for CO₂ conversion. Specifically, CH₂O is rarely identified as a stable majority product in CO₂ electroreduction (CO₂RR) due to its over reduction to methanol, limiting the feasibility of direct CO₂ electroconversion to formaldehyde for CO₂ sugars (7, 22). Alternatively, CH₂O can often be generated robustly in methanol oxidation and reports of methanol-producing electrocatalysts are more prevalent (7). Therefore, we targeted a multi-step catalytic strategy for CO₂ abiotic conversion to sugars through a route involving electroreduction to methanol followed by partial oxidation to formaldehyde (15). We aimed to achieve CO₂RR performance that would be suitable for generation of reduced C₁ species at a rate higher than photosynthetic CO₂ assimilation. As a first estimate we targeted partial current density j_{MeOH} on the order of 10 mA cm⁻² (which translates to 60 μmol methanol hr⁻¹ cm⁻², multiple factors higher than typical CO₂ fixation for a leaf of similar area) (23).

We selected cobalt phthalocyanine on carbon nanotube support (CoPc/CNT) as the CO₂RR catalyst due to growing consensus on its viability for generating beyond-two-electron C₁ products such as methanol (6 e) with moderate faradaic efficiency, a metric of electron utilization efficiency for a desired product (FE) (22, 24, 25). This unique activity is thought to be enabled by employing highly dispersed monomers of the catalyst strongly adsorbed to the CNT substrates, improving electron transfer kinetics and favoring strong CO binding and protonation (26). The single metal site helps ensure generation of single carbon products.

The CoPc/CNT catalyst can be straightforwardly synthesized by thorough co-sonication of the precursors in a suitable solvent, such as N,N-dimethylformamide. The catalyst immobilization and electrochemical activity was validated by electron microscopy, X-ray spectroscopy, and electrochemical characterizations. Notably, high-angle annular dark-field scanning transmission electron microscopy (HAADF-STEM) imaging identified the presence of single-atom cobalt sites with negligible aggregation and few powder crystallites remaining. X-ray absorption near-edge structure (XANES) displays the electronic fingerprint of adsorbed CoPc species as contrasted with the native CoPc precursor, especially the features at 7715 eV associated with molecular distortion caused by adsorption to the CNT substrate (25-27). While native CoPc shows negligible spectroscopic change under the CO₂RR at −0.6 V vs. RHE, operando XANES of CoPc/CNT shows a decrease in the white-line intensity at ~7725 eV, an increase in the pre-edge intensity at ~7710 eV and a slight absorption edge shifted to lower energy values, corresponding to the electroreduction of Co sites to a lower average oxidation state during CO₂RR. XPS analysis further indicated the presence of possible strain effects, indicated as N 1s peak splitting, induced by CoPc interaction with CNTs in the synthesized catalyst (26). These characterizations indicate an immobilized species that is chemically distinct from the native CoPc, which is known to terminate CO₂RR at CO (28).

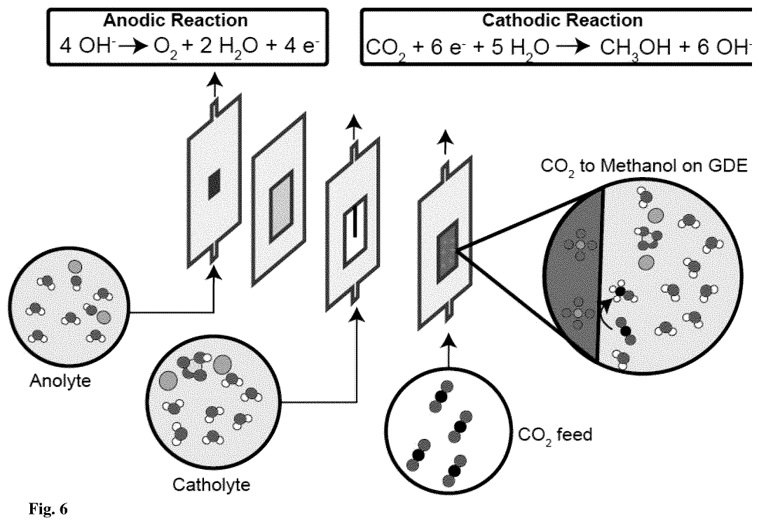

To assess overall performance in electrocatalysis, after validating the structure of the CoPc/CNT hybrid catalyst, we employed a gas diffusion electrode in a flow cell (FIG. 6) (29). Translating the CoPc/CNT system to a flow electrolysis system has so far been primarily limited to carbon monoxide reduction to overcome desorption of the weakly binding CO intermediate in convective conditions (25, 27, 30-32). However, prior generation of CO from CO₂ is an additional catalytic step that necessarily makes the modular process more complex. To achieve higher current density toward methanol directly from CO₂, we considered that prior work on the cascade reduction mechanism should indicate significant statistical effect of total catalyst loading. Higher catalyst loading improves FE_{MeOH} by increasing the likelihood for any desorbed CO product to find another CoPc site for further reduction. We explored two approaches: increased total mass loading of the CoPc/CNT ink and increased total dispersed loading of CoPc molecules on a given mass of CNT.

In the electrochemical flow cell using a gas diffusion electrode, we applied galvanostatic control to impose a reaction rate and screened a range of total current densities. Utilizing 0.55 mg cm⁻² of CoPc/CNT, we achieved a peak FE_{MeOH} of 27% at an average potential of −0.95 V vs. RHE, notably one of few reports of direct CO₂RR to methanol in the flow cell (25, 26, 30, 31, 33). Peak j_{MeOH} reaches 20 mA cm⁻² or 0.124 mmol methanol hr⁻¹ cm⁻². Previous work has emphasized that CoPc solubility in the loading step can be limiting for total catalyst loading on CNTs (27). Therefore, we doubled the mass loading of Co from 0.27 to 0.46 wt % on CNTs by two sequential CoPc loadings, which allowed for an improved peak j_{MeOH} of 35 mA cm⁻² at an average cathodic potential of approximately-1.09 V vs. RHE, (0.216 mmol hr⁻¹ cm⁻²), significantly exceeding the goal of 10 mA cm⁻². Additionally, formaldehyde is a direct product of CO₂RR on CoPc/CNT, consistent with previous findings (22). In our trials, FE_{CH20} does not exceed 5%, which is inadequate for conversion to sugars but suggests future potential for tuning immobilized molecular catalyst systems for direct CO₂ to CH₂O conversion. These results add to the growing body of knowledge that catalyst loading and associated kinetic factors govern the efficiency of a complex catalytic system such as CoPc/CNT (27, 30, 31, 33) and enables a path toward CO₂ conversion to reactive C₁ products for sugar generation.

### Photocatalytic Conversion of Methanol to Formaldehyde

After electrochemical production of methanol, conversion to the requisite formaldehyde for sugar synthesis must be achieved by exquisitely selective catalysis in order to minimize unnecessary material and efficiency losses and approximate the conversion that would be desired for direct CO₂RR. Methanol oxidation to formaldehyde is achievable via electrocatalysis; however, this process typically requires a high overpotential, resulting in low energy efficiency, and often employs expensive (anodized) noble metal catalysts (15, 34). Additionally, selectivity is not intrinsic to these electrocatalysts, and the required overpotential for CH₂O generation also allows for over-oxidation to undesired products such as CO and CO₂ (35). To address these challenges, we turned to photocatalytic methanol dehydrogenation (PMOR), which integrates methanol oxidation and the hydrogen evolution reaction (HER) in a single step, offering a sustainable method for processing the product stream from electrocatalysis.

Metal sulfide semiconductor photocatalysts are particularly interesting for this purpose due to their narrower bandgaps compared to metal oxides and efficient charge transport properties (36). Additionally, the valence band (VB) edges, which are primarily derived from sulfur 3p orbitals, are usually higher in energy, resulting in relatively soft oxidation ability (37). This can efficiently avoid the overoxidation of formaldehyde, therefore addressing the main issue raised by electrochemical methanol partial oxidation. Another advantage of utilizing metal sulfide semiconductors as photocatalysts is that most of them have a negative (vs. NHE) conduction band (CB) relative to TiO₂, suggesting strong reduction ability, which enables the utilization of an earth-abundant HER co-catalyst such as nickel (Ni) in addition to the typical noble platinum cocatalyst (38). This aligns with sustainability goals and indicates greater generality of the system for applications beyond PMOR.

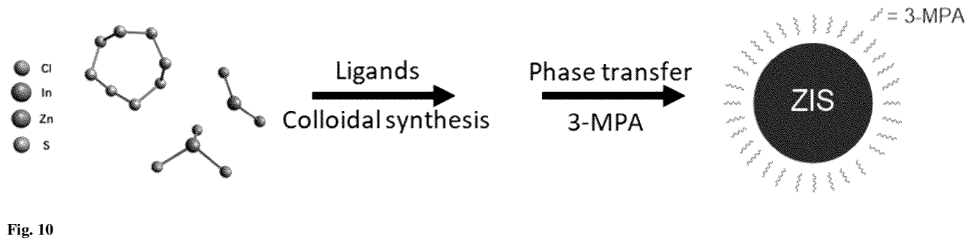

Within the class of metal sulfide photocatalysts, zinc indium sulfide (ZIS), a ternary chalcogenide, has attracted attention due to its low toxicity, suitable band edges for alcohol dehydrogenation, and high stability. However, ZIS synthesized by conventional hydrothermal or solvothermal methods often exhibits complex morphologies and low activity toward PMOR (39-41). Recent advancements in nanotechnology have highlighted the crucial role of nanocrystals in enhancing photocatalytic performance. Nanocrystals offer unique properties such as high surface area, quantum confinement effects, internal field enhancement, and tunable electronic structures, which can be exploited to improve the efficiency of photocatalytic reactions (42-44). To improve activity to a level suitable for CO₂ transformation of NHC sugars, we optimized the colloidal synthesis of ZIS by employing the hot injection of sulfur into a mixture of zinc and indium chloride precursors to form nanocrystals with well-defined crystalline morphology (FIG. 10) (45). The resulting ZIS NCs were characterized primarily as hexagonal nanoplates with a width of approximately 20 nm. High-resolution transmission electron microscopy (HRTEM) images, along with Fourier transform analysis, supported by powder X-ray diffraction (XRD) patterns, confirmed the hexagonal structure of ZIS at the atomic scale. The HRTEM image of ZIS shows the lattice parameter, a, of 3.9 Å which is consistent with theoretical values of 3.85 in the crystal model on the same zone axis.

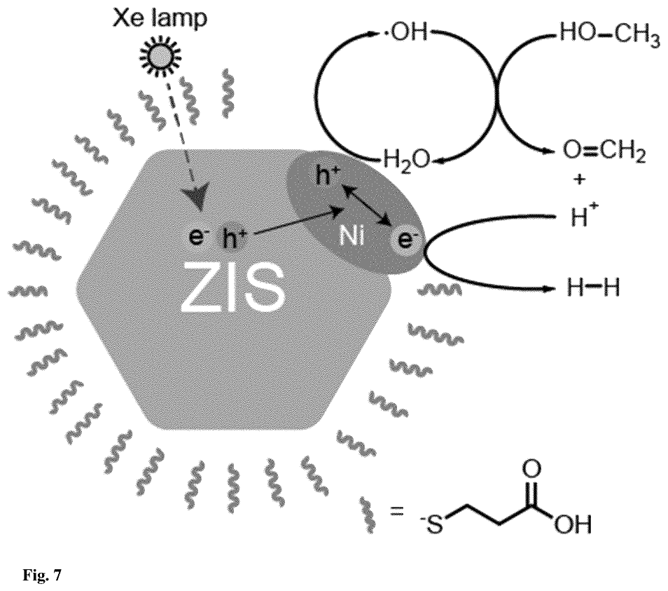

Surface modification of semiconductor photocatalysts with a co-catalyst significantly accelerates the HER process associated with photocatalytic alcohol dehydrogenation, thereby enhancing performance and stability of the photocatalysts (46). Ni, known for its high HER activity, low cost, and high abundance, was introduced onto our developed ZIS nanocrystals through an in situ photodeposition method. This method utilized methanol as a hole scavenger and NiCl₂ as a photoelectron acceptor, thereby directly introducing active Ni⁰ sites and avoiding their oxidation upon exposure to air (47). It should be noted that the CB minimum of our ZIS nanocrystals matches well with the reduction potential of the Ni²⁺. The resulting Ni/ZIS NCs exhibited uniform elemental distribution, as confirmed by STEM energy dispersive X-ray spectroscopy (STEM-EDX) mapping, indicating successful deposition of Ni. Post-deposition X-ray photoelectron spectroscopy (XPS) revealed the presence of Ni⁰, which may function as an active site for methanol dehydrogenation (FIG. 7) (48). This proposal was further validated by electron paramagnetic resonance (EPR) results, which showed strong signal enhancement of DMPO trapped hydroxyl radical (HO·) species in the presence of the Ni co-catalyst. The HO·radicals are expected reactive species generated on the nickel surface during the photocatalytic process, which further lead to the generation of formaldehyde through generation of the hydroxymethyl radical (·CH₂OH) (FIG. 7) (49, 50).

After successfully engineering catalyst nanostructure and cocatalyst loading, we investigated the catalyst performance in an air-free aqueous methanol solution. The Ni/ZIS NCs demonstrated excellent conversion rate and selectivity in catalytic trials of photocatalytic methanol dehydrogenation under simulated sunlight using a Xe lamp. Optimization of catalyst mass loading indicated an increasing trend in activity when the mass loading was reduced, leading to a productivity exceeding 80 mmol per gram photocatalyst per hour, with near-unit selectivity for formaldehyde. Decreased loading in suspension can prevent aggregation of nanoplates, improving the specific activity by allowing more NC surface area to be exposed. This indicates that a dilute dispersion of ZIS NCs is beneficial for the production of HCHO, which can be implemented in a large vessel without sacrificing its absolute production rate per hour and simultaneously minimizing catalyst utilization. To demonstrate improved production on a lab scale in a small tube, we used a more concentrated catalyst dispersion of 4 mg. In these conditions, 1.8 mmol of formaldehyde could be generated in 24 hours, making it suitable for NHC conversion to carbohydrates as it matches the scale used in our typical formoin batch reactions. For comparison, we also screened various metal sulfide and oxide structures under similar conditions to evaluate their PMOR activity. The activity of the Ni/ZIS NCs was superior, particularly compared to other sulfides. Hydrothermally synthesized Ni/ZIS was only one-fourth as active, highlighting the benefit of our nanoengineering approach in enhancing photocatalytic efficiency. For a potentially photosynthetic pathway of CO₂ to sugars, the ZIS NCs are thus an excellent component to fill in the technology gap of direct CO₂ conversion to formaldehyde, allowing multiple handles for tunability that would be suitable to treat the engineering task of post-electrolysis methanol conversion in a sustainable manner.

NHC-Catalyzed Formoin Condensation for Selective Carbohydrate Generation from Formaldehyde

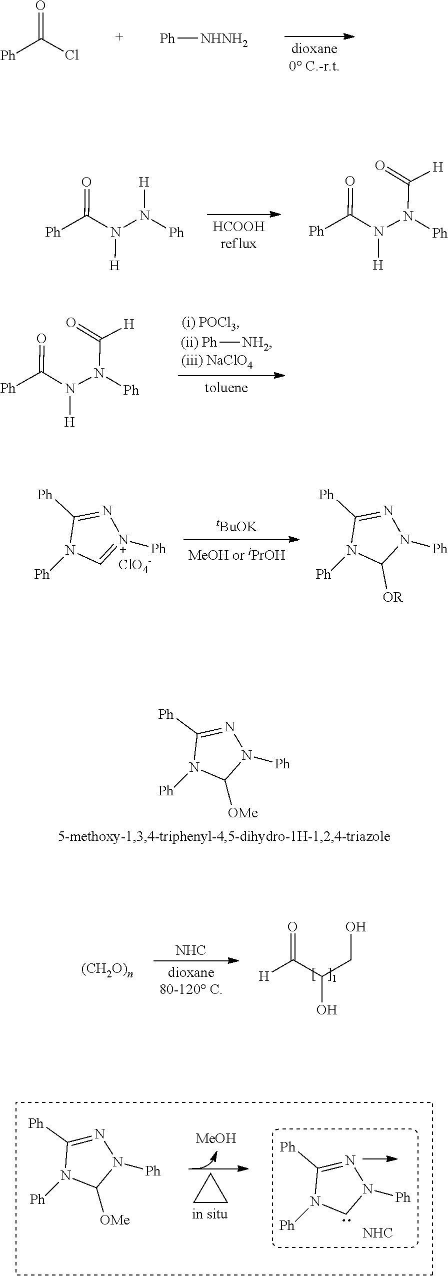

TABLE 1
Some conditions and selectivities and carbon yields of NHC trials for carbohydrate
synthesis from formaldehyde, including comparisons with abiotic formose reactions.

| | Time | Selectivity C3 | Selectivity C4 | Selectivity C5 | Selectivity C6 | Yield |
|---|---|---|---|---|---|---|
| Entry | (h) | (%) | (%) | (%) | (%) | (%) |
| 1 | 3 | 0 | 84 | 16 | 0 | #N/A |
| 2^{a} | 3 | 0 | 37 | 57 | 5 | 103 |
| 3^{b, c, d,} | 3 | 0 | 23 | 47 | 30 | 67 |
| 4^{e, f} | 0.5 | 25 | 13 | 33 | 28 | 36 |
| 5^{g, f} | 0.5 | 43 | 24 | 22 | 12 | 44 |
| 6^{d, h} | 2 | 25 | 73 | 3 | 0 | 92 |
| 7^{i, j} | 1 | 6 | 27 | 46 | 20 | 33 |
| 8^{i, k} | 1 | 10 | 30 | 16 | 45 | 17 |
| 9^{l} | 3 | 36 | 62 | 2 | 0 | 52 |
| 10^{m, d} | 3 | 0 | 27 | 68 | 4 | 9 |
| 11^{n, d} | 3 | 0 | 67 | 30 | 3 | 105 |

Conditions: HCHO (60 mg, 2 mmol), NHC-1 (3 mg, 0.01 mmol), 80° C., dioxane (5 mL), yield was estimated on carbon basis.
^{a}90° C.
^{b}dioxane (2.5 mL)
^{c}NHC-1 (9 mg, 0.03 mmol).
^{d}100° C.
^{e}Classic Formose conditions: 1M HCHO, 5 mL H₂O, pH 11 by Ca(OH)₂, 80° C., 100 μM glycolaldehyde.
^{f}HCHO (120 mg, 4 mmol).
^{g}Air company: 0.87M HCHO, 10% methanol v/v in 5 mL water, 60° C., 0.1 g Ca(OH)₂, 0.1 g L-proline.
^{h}NHC-2 (3 mg, 0.01 mmol).
ⁱMethanol (4 mL) as solvent.
^{j}MORose Analogue: 0.4M HCHO in 90% MeOH/10% H₂O v/v, 80° C., 100 μM glycolaldehyde, 0.1M NaClO₄, 40 mM Sr(OH)₂.
^{k}Methanolic Formose: 0.4M HCHO in 90% MeOH/10% H₂O v/v, 80° C., 100 μM glycolaldehyde, 40 mM Sr(OH)₂.
^{l}DMF (5 mL) as solvent.
^{m}Methanol (5 mL) as solvent.
ⁿDioxane (5 mL) and methanol (0.1 mL) as solvent.

### REFERENCES AND NOTES

1. P. De Luna, C. Hahn, D. Higgins, S. A. Jaffer, T. F. Jaramillo, E. H. Sargent, What would it take for renewably powered electrosynthesis to displace petrochemical processes? Science 364, eaav3506 (2019).
2. F. Dinger, U. Platt, Towards an Artificial Carbohydrates Supply on Earth. Front. Sustain. Food Syst. 4 (2020).
3. J. Poore, T. Nemecek, Reducing food's environmental impacts through producers and consumers. Science 360, 987-992 (2018).
4. N. Yan, K. Zhou, Y. W. Tong, D. T. Leong, M. P. Dickieson, Pathways to food from CO₂ via ‘green chemical farming.’ Nat. Sustain. 5, 907-909 (2022).
5. L. T. Mika, E. Cséfalvay, Á. Németh, Catalytic Conversion of Carbohydrates to Initial Platform Chemicals: Chemistry and Sustainability. Chem. Rev. 118, 505-613 (2018).
6. T. Cai, H. Sun, J. Qiao, L. Zhu, F. Zhang, J. Zhang, Z. Tang, X. Wei, J. Yang, Q. Yuan, W. Wang, X. Yang, H. Chu, Q. Wang, C. You, H. Ma, Y. Sun, Y. Li, C. Li, H. Jiang, Q. Wang, Y. Ma, Cell-free chemoenzymatic starch synthesis from carbon dioxide. Science 373, 1523-1527 (2021).
7. S. Cestellos-Blanco, S. Louisia, M. B. Ross, Y. Li, N. E. Soland, T. C. Detomasi, J. N. Cestellos Spradlin, D. K. Nomura, P. Yang, Toward abiotic sugar synthesis from CO₂ electrolysis. Joule 6, 2304-2323 (2022).
8. H. Tang, L. Wu, S. Guo, W. Cao, W. Ma, X. Wang, J. Shen, M. Wang, Q. Zhang, M. Huang, X. Luo, J. Zeng, J. D. Keasling, T. Yu, Metabolic engineering of yeast for the production of carbohydrate-derived foods and chemicals from C₁-3 molecules. Nat. Catal. 7, 21-34 (2024).
9. G. Liu, Y. Zhong, Z. Liu, G. Wang, F. Gao, C. Zhang, Y. Wang, H. Zhang, J. Ma, Y. Hu, A. Chen, J. Pan, Y. Min, Z. Tang, C. Gao, Y. Xiong, Solar-driven sugar production directly from CO₂ via a customizable electrocatalytic-biocatalytic flow system. Nat. Commun. 15, 2636 (2024).
10. J. Yang, W. Song, T. Cai, Y. Wang, X. Zhang, W. Wang, P. Chen, Y. Zeng, C. Li, Y. Sun, Y. Ma, De novo artificial synthesis of hexoses from carbon dioxide. Sci. Bull. 68, 2370-2381 (2023).
11. E. C. Hann, S. Overa, M. Harland-Dunaway, A. F. Narvaez, D. N. Le, M. L. Orozco-Cárdenas, F. Jiao, R. E. Jinkerson, A hybrid inorganic-biological artificial photosynthesis system for energy-efficient food production. Nat. Food 3, 461-471 (2022).
12. R. E. Blankenship, D. M. Tiede, J. Barber, G. W. Brudvig, G. Fleming, M. Ghirardi, M. R. Gunner, W. Junge, D. M. Kramer, A. Melis, T. A. Moore, C. C. Moser, D. G. Nocera, A. J. Nozik, D. R. Ort, W. W. Parson, R. C. Prince, R. T. Sayre, Comparing Photosynthetic and Photovoltaic Efficiencies and Recognizing the Potential for Improvement. Science 332, 805-809 (2011).
13. J. Barber, Photosynthetic energy conversion: natural and artificial. Chem. Soc. Rev. 38, 185-196 (2008).
14. D. Leger, S. Matassa, E. Noor, A. Shepon, R. Milo, A. Bar-Even, Photovoltaic-driven microbial protein production can use land and sunlight more efficiently than conventional crops. Proc. Natl. Acad. Sci. 118, e2015025118 (2021).
15. N. E. Soland, I. Roh, W.-S. Huynh, P. Yang, Synthesis of Carbohydrates from Methanol Using Electrochemical Partial Oxidation over Palladium with the Integrated Formose Reaction. ACS Sustain. Chem. Eng. 11, 12478-12483 (2023).
16. Md. Z. Iqbal, S. Novalin, Analysis of formose sugar and formaldehyde by high-performance liquid chromatography. J. Chromatogr. A 1216, 5116-5121 (2009).
17. D. Zhang, C. Jarava-Barrera, S. Bontemps, Selective Reductive Dimerization of CO₂ into Glycolaldehyde. ACS Catal. 11, 4568-4575 (2021).
18. J. Henrique Teles, J.-P. Melder, K. Ebel, R. Schneider, E. Gehrer, W. Harder, S. Brode, D. Enders, K. Breuer, G. Raabe, The Chemistry of Stable Carbenes. Part 2. Benzoin-type condensations of formaldehyde catalyzed by stable carbenes. Helv. Chim. Acta 79, 61-83 (1996).
19. N. Marion, S. Díez-González, S. P. Nolan, N-Heterocyclic Carbenes as Organocatalysts. Angew. Chem. Int. Ed. 46, 2988-3000 (2007).
20. J. Castells, F. Geijo, F. López-Calahorra, The “formoin reaction”: A promising entry to carbohydrates from formaldehyde. Tetrahedron Lett. 21, 4517-4520 (1980).
21. D. Enders, K. Breuer, G. Raabe, J. Runsink, J. H. Teles, J.-P. Melder, K. Ebel, S. Brode, Preparation, Structure, and Reactivity of 1,3,4-Triphenyl-4,5-dihydro-1H-1,2,4-triazol-5-ylidene, a New Stable Carbene. Angew. Chem. Int. Ed. Engl. 34, 1021-1023 (1995).
22. E. Boutin, A. Salamé, L. Merakeb, T. Chatterjee, M. Robert, On the Existence and Role of Formaldehyde During Aqueous Electrochemical Reduction of Carbon Monoxide to Methanol by Cobalt Phthalocyanine. Chem. —Eur. J. 28, e202200697 (2022).
23. Z. Zhou, P. Su, X. Wu, R. Shi, X. Ding, Leaf and Community Photosynthetic Carbon Assimilation of Alpine Plants Under in-situ Warming. Front. Plant Sci. 12 (2021).
24. Y. Wu, Z. Jiang, X. Lu, Y. Liang, H. Wang, Domino electroreduction of CO₂ to methanol on a molecular catalyst. Nature 575, 639-642 (2019).
25. X. Ren, J. Zhao, X. Li, J. Shao, B. Pan, A. Salamé, E. Boutin, T. Groizard, S. Wang, J. Ding, X. Zhang, W.-Y. Huang, W.-J. Zeng, C. Liu, Y. Li, S.-F. Hung, Y. Huang, M. Robert, B. Liu, In-situ spectroscopic probe of the intrinsic structure feature of single-atom center in electrochemical CO/CO2 reduction to methanol. Nat. Commun. 14, 3401 (2023).
26. J. Su, C. B. Musgrave, Y. Song, L. Huang, Y. Liu, G. Li, Y. Xin, P. Xiong, M. M.-J. Li, H. Wu, M. Zhu, H. M. Chen, J. Zhang, H. Shen, B. Z. Tang, M. Robert, W. A. Goddard, R. Ye, Strain enhances the activity of molecular electrocatalysts via carbon nanotube supports. Nat. Catal. 6, 818-828 (2023).
27. C. L. Rooney, M. Lyons, Y. Wu, G. Hu, M. Wang, C. Choi, Y. Gao, C.-W. Chang, G. W. Brudvig, Z. Feng, H. Wang, Active Sites of Cobalt Phthalocyanine in Electrocatalytic CO₂ Reduction to Methanol. Angew. Chem. Int. Ed. 63, e202310623 (2024).
28. M. Wang, K. Torbensen, D. Salvatore, S. Ren, D. Joulié, F. Dumoulin, D. Mendoza, B. Lassalle-Kaiser, U. Işci, C. P. Berlinguette, M. Robert, CO₂ electrochemical catalytic reduction with a highly active cobalt phthalocyanine. Nat. Commun. 10, 3602 (2019).
29. C. P. O'Brien, R. K. Miao, A. Shayesteh Zeraati, G. Lee, E. H. Sargent, D. Sinton, CO₂ Electrolyzers. Chem. Rev. 124, 3648-3693 (2024).
30. L. Yao, K. E. Rivera-Cruz, P. M. Zimmerman, N. Singh, C. C. L. McCrory, Electrochemical CO₂ Reduction to Methanol by Cobalt Phthalocyanine: Quantifying CO₂ and CO Binding Strengths and Their Influence on Methanol Production. ACS Catal. 14, 366-372 (2024).
31. J. Li, B. Shang, Y. Gao, S. Cheon, C. L. Rooney, H. Wang, Mechanism-guided realization of selective carbon monoxide electroreduction to methanol. Nat. Synth. 2, 1194-1201 (2023).
32. T. Chan, C. J. Kong, A. J. King, F. Babbe, R. R. Prabhakar, C. P. Kubiak, J. W. Ager, Role of Mass Transport in Electrochemical CO2 Reduction to Methanol Using Immobilized Cobalt Phthalocyanine. ACS Appl. Energy Mater. 7, 3091-3098 (2024).
33. T. Chan, C. Kong, A. King, R. Prabhakar, F. Babbe, C. Kubiak, J. Ager, Role of Mass Transport in Electrochemical CO₂ Reduction to Methanol Using Immobilized Cobalt Phthalocyanine. ChemRxiv [Preprint] (2023). https://doi.org/10.26434/chemrxiv-2023-cqn7d.
34. J. Baessler, T. Oliveira, R. Keller, M. Wessling, Paired Electrosynthesis of Formic Acid from CO₂ and Formaldehyde from Methanol. ACS Sustain. Chem. Eng. 11, 6822-6828 (2023).
35. T. Iwasita, Electrocatalysis of methanol oxidation. Electrochimica Acta (2002).
36. Q. Zhu, Q. Xu, M. Du, X. Zeng, G. Zhong, B. Qiu, J. Zhang, Recent Progress of Metal Sulfide Photocatalysts for Solar Energy Conversion. Adv. Mater. 34, 2202929 (2022).
37. X. Wu, S. Xie, H. Zhang, Q. Zhang, B. F. Sels, Y. Wang, Metal Sulfide Photocatalysts for Lignocellulose Valorization. Adv. Mater. 33, 2007129 (2021).
38. J. Ran, J. Zhang, J. Yu, M. Jaroniec, S. Z. Qiao, Earth-abundant cocatalysts for semiconductor-based photocatalytic water splitting. Chem. Soc. Rev. 43, 7787-7812 (2014).
39. S. Meng, H. Wu, Y. Cui, X. Zheng, H. Wang, S. Chen, Y. Wang, X. Fu, One-step synthesis of 2D/2D-3D NiS/Zn3In2S6 hierarchical structure toward solar-to-chemical energy transformation of biomass-relevant alcohols. Appl. Catal. B Environ. 266, 118617 (2020).
40. T. Zhang, T. Wang, F. Meng, M. Yang, S. Kawi, Recent advances in ZnIn2S4-based materials towards photocatalytic purification, solar fuel production and organic transformations. J. Mater. Chem. C 10, 5400-5424 (2022).
41. Y. Wang, Y. Zhang, X. Xin, J. Yang, M. Wang, R. Wang, P. Guo, W. Huang, A. J. Sobrido, B. Wei, X. Li, In situ photocatalytically enhanced thermogalvanic cells for electricity and hydrogen production. Science 381, 291-296 (2023).
42. S. Cestellos-Blanco, H. Zhang, J. M. Kim, Y. Shen, P. Yang, Photosynthetic semiconductor biohybrids for solar-driven biocatalysis. Nat. Catal. 3, 245-255 (2020).
43. L. Cheng, Q. Xiang, Y. Liao, H. Zhang, CdS-Based photocatalysts. Energy Environ. Sci. 11, 1362-1391 (2018).
44. J. Wang, Y. Shi, Y. Wang, Z. Li, Rational Design of Metal Halide Perovskite Nanocrystals for Photocatalytic CO2 Reduction: Recent Advances, Challenges, and Prospects. ACS Energy Lett. 7, 2043-2059 (2022).
45. B. Kempken, V. Dzhagan, D. R. T. Zahn, M. J. P. Alcocer, I. Kriegel, F. Scotognella, J. Parisi, J. Kolny-Olesiak, Synthesis, optical properties, and photochemical activity of zinc-indium-sulfide nanoplates. RSC Adv. 5, 89577-89585 (2015).
46. J. Yang, D. Wang, H. Han, C. Li, Roles of Cocatalysts in Photocatalysis and Photoelectrocatalysis. Acc. Chem. Res. 46, 1900-1909 (2013).
47. K. Wenderich, G. Mul, Methods, Mechanism, and Applications of Photodeposition in Photocatalysis: A Review. Chem. Rev. 116, 14587-14619 (2016).
48. Z. Chai, T.-T. Zeng, Q. Li, L.-Q. Lu, W.-J. Xiao, D. Xu, Efficient Visible Light-Driven Splitting of Alcohols into Hydrogen and Corresponding Carbonyl Compounds over a Ni-Modified CdS Photocatalyst. J. Am. Chem. Soc. 138, 10128-10131 (2016).
49. J. Schneider, D. W. Bahnemann, Undesired Role of Sacrificial Reagents in Photocatalysis. J. Phys. Chem. Lett. 4, 3479-3483 (2013).
50. Z. Gao, N. Luo, Z. Huang, S. H. Taylor, F. Wang, Controlling Radical Intermediates in Photocatalytic Conversion of Low-Carbon-Number Alcohols. ACS Sustain. Chem. Eng. 9, 6188-6202 (2021).

### ADDITIONAL REFERENCES

ADDIN ZOTERO_BIBL {“uncited”: [ ], “omitted”: [ ], “custom”: [ ]} CSL_BIBLIOGRAPHY 1. R. E. Blankenship, D. M. Tiede, J. Barber, G. W. Brudvig, G. Fleming, M. Ghirardi, M. R. Gunner, W. Junge, D. M. Kramer, A. Melis, T. A. Moore, C. C. Moser, D. G. Nocera, A. J. Nozik, D. R. Ort, W. W. Parson, R. C. Prince, R. T. Sayre, Comparing Photosynthetic and Photovoltaic Efficiencies and Recognizing the Potential for Improvement. Science 332, 805-809 (2011).
2. S. Cestellos-Blanco, S. Louisia, M. B. Ross, Y. Li, N. E. Soland, T. C. Detomasi, J. N. Cestellos Spradlin, D. K. Nomura, P. Yang, Toward abiotic sugar synthesis from CO₂ electrolysis. Joule 6, 2304-2323 (2022).
3. T. Cai, H. Sun, J. Qiao, L. Zhu, F. Zhang, J. Zhang, Z. Tang, X. Wei, J. Yang, Q. Yuan, W. Wang, X. Yang, H. Chu, Q. Wang, C. You, H. Ma, Y. Sun, Y. Li, C. Li, H. Jiang, Q. Wang, Y. Ma, Cell-free chemoenzymatic starch synthesis from carbon dioxide. Science 373, 1523-1527 (2021).
4. C. P. O'Brien, R. K. Miao, A. Shayesteh Zeraati, G. Lee, E. H. Sargent, D. Sinton, CO2 Electrolyzers. Chem. Rev. 124, 3648-3693 (2024).
5. C. L. Rooney, M. Lyons, Y. Wu, G. Hu, M. Wang, C. Choi, Y. Gao, C.-W. Chang, G. W. Brudvig, Z. Feng, H. Wang, Active Sites of Cobalt Phthalocyanine in Electrocatalytic CO₂ Reduction to Methanol. Angew. Chem. Int. Ed. 63, e202310623 (2024).
6. X. Ren, J. Zhao, X. Li, J. Shao, B. Pan, A. Salamé, E. Boutin, T. Groizard, S. Wang, J. Ding, X. Zhang, W.-Y. Huang, W.-J. Zeng, C. Liu, Y. Li, S.-F. Hung, Y. Huang, M. Robert, B. Liu, In-situ spectroscopic probe of the intrinsic structure feature of single-atom center in electrochemical CO/CO₂ reduction to methanol. Nat. Commun. 14, 3401 (2023).
7. J. Su, C. B. Musgrave, Y. Song, L. Huang, Y. Liu, G. Li, Y. Xin, P. Xiong, M. M.-J. Li, H. Wu, M. Zhu, H. M. Chen, J. Zhang, H. Shen, B. Z. Tang, M. Robert, W. A. Goddard, R. Ye, Strain enhances the activity of molecular electrocatalysts via carbon nanotube supports. Nat. Catal. 6, 818-828 (2023).

1. A method for selective generation of a sugar from carbon dioxide, comprising:
(a) catalytically converting carbon dioxide (CO₂) to formaldehyde (CH₂O) via (i) reduction of CO₂ to methanol (CH₃OH) followed by oxidation to CH₂O, or (ii) direct reduction of CO₂ to CH₂O; and
(b) using an N-heterocyclic carbene catalyst to selectively condense the formaldehyde to generate the sugar.

2. The method of claim 1 wherein step (a) comprises electrochemical reduction of the CO₂ to methanol in an electrochemical carbon dioxide reduction reaction (CO₂RR), and photochemical oxidation of the methanol to formaldehyde in a photooxidation of methanol to formaldehyde oxidation reaction (PMOR).

3. The method of claim 1, wherein step (a) comprises electrochemical reduction of the CO₂ to methanol on a catalyst comprising carbon nanotube (CNT)-supported cobalt (II) phthalocyanine (CoPc), optionally deposited on a support, such as carbon paper, in aqueous or nonaqueous (e.g. acetonitrile) solvent with supporting electrolyte.

4. The method of claim 1, wherein step (a) comprises electrochemical reduction of the CO₂ to methanol using a membrane electrode assembly (MEA) flow cell architecture, such as a gas diffusion electrode to enhance the transport of CO₂ to the catalyst for increased product yield.

5. The method of claim 1, wherein step (a) comprises electrochemical reduction of the CO₂ to methanol using an H-cell architecture to increase a faradaic efficiency.

6. The method of claim 1, wherein step (a) comprises partial oxidation of CH₃OH to CH₂O using a noble metal catalysts, such as platinum and palladium, capable of electrooxidation of CH₃OH, and optionally, tuning reaction parameters, including methanol content, to improve selectivity to CH₂O over products such as CO and CO₂, improving space-time yield by utilizing a flow cell architecture which enhances the convective removal of formaldehyde product from the electrode surface.

7. The method of claim 1, wherein step (a) comprises electrochemical reduction of the CO₂ to methanol, wherein faradaic efficiency, a metric describing the fraction of electric current utilized for generation of the desired product, exceeds 50% in an H-cell configuration on palladium foil.

8. The method of claim 1, wherein step (a) comprises electrochemical reduction of the CO₂ to methanol, wherein partial current densities to formaldehyde approach the milliamp per square centimeter range depending on overpotential applied, methanol concentration and solvent composition, solution pH, convective conditions in the cell, and electrode pretreatment.

9. The method of claim 1, wherein step (a) comprises electrochemical reduction of the CO₂ to methanol (CH₃OH) followed by oxidation to CH₂O, wherein photocatalytic oxidation of CH₃OH to CH₂O comprises selecting photocatalysts with suitable band edge positions and stability for extended operation, optionally in an implementation utilizing zinc-indium-sulfide (ZIS) nanostructures under UV or broad-spectrum irradiance.

10. The method of claim 1, wherein step (a) comprises electrochemical reduction of the CO₂ to methanol to yield reaction products including hydrogen (H₂), carbon monoxide (CO), CH₂O, and methanol (CH₃OH), the method further comprising adjusting relative selectivity and yield of these products by tuning catalyst loading, solution pH, supporting electrolyte, gas feed rate, applied potential, temperature, and electrolyzer design.

11. The method of claim 1, wherein step (b) comprises preactivated NHC in an aprotic, nonpolar solvent such as dioxane, wherein there is little driving force for ketose formation via isomerization, and the condensation reaction can proceed until ring formation terminates the reaction differentially based on reaction temperature and composition, wherein higher order carbohydrates are afforded without byproducts from the classical formose reaction.

12. The method of claim 1, wherein in step (b) NHC formose catalysis is tuned further by employing a diverse range of NHCs, including enantiomerically selective catalysis more stability afforded by modifying the steric hindrance at the carbene center, allowing for implementation in aqueous solvent; or more reactivity is afforded by replacing the substituents;
or enhanced product separation is achieved by immobilization strategies of the catalysts onto polymers or silica gel.

13. The method of claim 1, wherein step (a) comprises direct reduction of CO₂ to CH₂O using a boron doped diamond catalyst, preferably utilizing an aqueous or mixed nonaqueous (e.g. acetonitrile/water) electrolyte in an H-cell or membrane electrode assembly (MEA) flow cell architecture, such as a gas diffusion electrode, for direct electrosynthesis of CH₂O from CO₂.

14. The method of claim 1, wherein step (a) provides a partial oxidation faradaic efficiency exceeding 50%.

15. The method of claim 1, wherein selectivity for the formaldehyde-to-sugar transformation exceeds 80% toward desired pentose, tetrose, triose, and hexose products.

16. The method of claim 1, wherein the sugar is a C₃-C₆ aldose.

17. The method of claim 1, wherein step (b) generates the sugar without oxidized byproducts from a classical formose reaction.

18. The method of claim 1, wherein step (b) comprises organocatalytic benzoin-like condensation of formaldehyde catalyzed by N-heterocyclic carbene (NHC) to preferentially form aldose and unbranched monosaccharides, distinguishing this approach from uncontrolled alkaline formose.

19. The method of claim 1, comprising in situ generation and utilization of the formaldehyde to minimize processing steps.

20. The method of claim 1, further comprising isolating the sugar.
